(19) United States
(12) Patent Application Publication
(10) Pub. No.: US 2024/0341254 A1
(43) Pub. Date: Oct. 17, 2024

(54) VARIN PROFILES

(71) Applicant: Phylos Bioscience, Inc., Portland, OR (US)

(72) Inventors: Jared Reynbery, Portland, OR (US); Erica Bakker, Portland, OR (US); John McFerson, Portland, OR (US); Alisha Holloway, Portland, OR (US)

(73) Assignee: Phylos Bioscience, Inc., Portland, OR (US)

(21) Appl. No.: 18/701,037

(22) PCT Filed: Oct. 12, 2022
(86) PCT No.: PCT/US2022/078000

§ 371 Date: Apr. 12, 2024

Related U.S. Application Data
(60) Provisional application No. 63/255,883, filed on Oct. 14, 2021.

(51) Int. Cl. A01H 6/28; A01H 5/02

(52) U.S. Cl. CPC A01H 6/28; A01H 5/02

(57) ABSTRACT

The present invention discloses Cannabis plants, seeds and other plant parts, tissue cultures, and products having elevated levels of varin molecules, including tetrahydrocannabivarin (THCV) and/or cannabidivarin (CBDV). Each of THCV and CBDV have unique pharmacological profiles with distinct molecular targets, and can be used as agents for antiepileptic or anticonvulsant activity, obesity-associated glucose intolerance, appetite suppression, anxiety management for PTSD, diabetic neuropathy, or major neuropathic and pain related pathologies.

### CROSS REFERENCE TO RELATED APPLICATIONS

This application claims priority benefit to U.S. provisional application No. 63/255,883, filed Oct. 14, 2021, the entire contents of which are hereby incorporated by reference.

### BACKGROUND OF THE INVENTION

Cannabis plants contain over a hundred known cannabinoids, which bind to endogenous endocannabinoid receptors. Varinolic cannabinoids, as known as varins, are a type of cannabinoid compounds having three carbon atoms in their alkyl side chain instead of the five carbon atom alkyl side chains more commonly associated with cannabinoids. Two such varins are tetrahydrocannabivarin (THCV) and cannabidivarin (CBDV), which are homologues of tetrahydrocannabinol (THC) and cannabidiol (CBD), respectively. Each varin has a unique pharmacological profile and distinct molecular targets.

THCV and CBDV have potential benefits across a broad set of applications. Cannabis strains or extracts with high THCV levels, for example, can be used as an agent for anticonvulsant activity, obesity-associated glucose intolerance, appetite suppression, anxiety management for PTSD, diabetic neuropathy, and major neuropathic and pain related pathologies. Another THCV application is that of an appetite suppressing compound. CBDV has been shown to have anti-epileptic and anticonvulsant activity.

Research and development as well as the sale of varin products has been limited due to low commonly occurring levels of varins in Cannabis flower. The ability to produce Cannabis with high varin levels will create a platform for a new Cannabinoid category with differentiated, high margin products in both medical and recreational markets. The present invention solves these problems by providing novel Cannabis plants and plant parts having high levels of THCV, CBDV, or THCV combined with CBDV, which are useful for medicinal and/or recreational purposes.

### SUMMARY OF THE INVENTION

The present teachings relate to Cannabis plants, parts, and products having elevated varins.

In an embodiment, a Cannabis plant is provided that comprises a tetrahydrocannabivarin (“THCV”) amount of between 5 and 25%. In an embodiment, the THCV amount is one or more of between 5% and 24%, 5% and 23%, 5% and 22%, 5% and 21%, 5% and 20%, 5% and 19%, 5% and 18%, 5% and 17%, 5% and 16%, 5% and 15%, 5% and 14%, 5% and 13%, 5% and 12%, 5% and 11%, 5% and 10%, 5% and 9%, 5% and 8%, 5% and 7%, 5% and 6%, 6% and 25%, 6% and 24%, 6% and 23%, 6% and 22%, 6% and 21%, 6% and 20%, 6% and 19%, 6% and 18%, 6% and 17%, 6% and 16%, 6% and 15%, 6% and 14%, 6% and 13%, 6% and 12%, 6% and 11%, 6% and 10%, 6% and 9%, 6% and 8%, 6% and 7%, 7% and 25%, 7% and 24%, 7% and 23%, 7% and 22%, 7% and 21%, 7% and 20%, 7% and 19%, 7% and 18%, 7% and 17%, 7% and 16%, 7% and 15%, 7% and 14%, 7% and 13%, 7% and 12%, 7% and 11%, 7% and 10%, 7% and 9%, 7% and 8%, 8% and 25%, 8% and 24%, 8% and 23%, 8% and 22%, 8% and 21%, 8% and 20%, 8% and 19%, 8% and 18%, 8% and 17%, 8% and 16%, 8% and 15%, 8% and 14%, 8% and 13%, 8% and 12%, 8% and 11%, 8% and 10%, 8% and 9%, 9% and 25%, 9% and 24%, 9% and 23%, 9% and 22%, 9% and 21%, 9% and 20%, 9% and 19%, 9% and 18%, 9% and 17%, 9% and 16%, 9% and 15%, 9% and 14%, 9% and 13%, 9% and 12%, 9% and 11%, 9% and 10%, 10% and 25%, 10% and 24%, 10% and 23%, 10% and 22%, 10% and 21%, 10% and 20%, 10% and 19%, 10% and 18%, 0% and 17%, 10% and 16%, 10% and 15%, 10% and 14%, 10% and 13%, 10% and 12%, 10% and 11%, 11% and 25%, 11% and 24%, 11% and 23%, 11% and 22%, 11% and 21%, 11% and 20%, 11% and 19%, 11% and 18%, 11% and 17%, 11% and 16%, 11% and 15%, 11% and 14%, 11% and 13%, 11% and 12%, 12% and 25%, 12% and 24%, 12% and 23%, 12% and 22%, 12% and 21%, 12% and 20%, 12% and 19%, 12% and 18%, 12% and 17%, 12% and 16%, 12% and 15%, 12% and 14%, 12% and 13%, 13% and 25%, 13% and 24%, 13% and 23%, 13% and 22%, 13% and 21%, 13% and 20%, 13% and 19%, 13% and 18%, 13% and 17%, 13% and 16%, 13% and 15%, 13% and 14%, 14% and 25%, 14% and 24%, 14% and 23%, 14% and 22%, 14% and 21%, 14% and 20%, 14% and 19%, 14% and 18%, 14% and 17%, 14% and 16%, 14% and 15%, 15% and 25%, 15% and 24%, 15% and 23%, 15% and 22%, 15% and 21%, 15% and 20%, 15% and 19%, 15% and 18%, 15% and 17%, 15% and 16%, 16% and 25%, 16% and 24%, 16% and 23%, 16% and 22%, 16% and 21%, 16% and 20%, 16% and 19%, 16% and 18%, 16% and 17%, 17% and 25%, 17% and 24%, 17% and 23%, 17% and 22%, 17% and 21%, 17% and 20%, 17% and 19%, 17% and 18%, 18% and 25%, 18% and 24%, 18% and 23%, 18% and 22%, 18% and 21%, 18% and 20%, 18% and 19%, 19% and 25%, 19% and 24%, 19% and 23%, 19% and 22%, 19% and 21%, 19% and 20%, 20% and 25%, 20% and 24%, 20% and 23%, 20% and 22%, 20% and 21%, 21% and 25%, 21% and 24%, 21% and 23%, 21% and 22%, 22% and 25%, 22% and 24%, 22% and 23%, 23% and 25%, 23% and 24%, or 24% and 25%. In another embodiment, a seed of the Cannabis plants as described herein is provided. In an embodiment, the seed further comprises a trait introduced by backcrossing or genetic transformation. In an embodiment, a Cannabis plant, or part thereof, is provided that includes at least one plant cell, produced by growing the seed. In another embodiment, an F1 hybrid seed produced by crossing the Cannabis plants as described with a different Cannabis plant is provided. In an embodiment, a plant or plant part is provided that is grown from the F1 hybrid seed that comprises at least one cell of the F1 hybrid plant. In another embodiment, a tissue culture of cells produced from the Cannabis plants as described herein is provided. In an embodiment, a plant generated from the tissue culture is provided. In an embodiment, a protoplast produced from the Cannabis plants as described herein is provided. In another embodiment, a Cannabis product produced from the Cannabis plants as described herein is provided. In an embodiment, a method of generating a Cannabis product is provided. In an embodiment, the product is a kief, hashish, bubble hash, an edible product, flower, seed, solvent reduced oil, sludge, e-juice, or tincture.

In an embodiment, a Cannabis plant is provided that comprises a cannabidivarin (“CBDV”) amount of between 5 and 25%. In an embodiment, the CBDV amount is one or more of between 5% and 24%, 5% and 23%, 5% and 22%, 5% and 21%, 5% and 20%, 5% and 19%, 5% and 18%, 5% and 17%, 5% and 16%, 5% and 15%, 5% and 14%, 5% and 13%, 5% and 12%, 5% and 11%, 5% and 10%, 5% and 9%, 5% and 8%, 5% and 7%, 5% and 6%, 6% and 25%, 6% and 24%, 6% and 23%, 6% and 22%, 6% and 21%, 6% and 20%, 6% and 19%, 6% and 18%, 6% and 17%, 6% and 16%, 6% and 15%, 6% and 14%, 6% and 13%, 6% and 12%, 6% and 11%, 6% and 10%, 6% and 9%, 6% and 8%, 6% and 7%, 7% and 25%, 7% and 24%, 7% and 23%, 7% and 22%, 7% and 21%, 7% and 20%, 7% and 19%, 7% and 18%, 7% and 17%, 7% and 16%, 7% and 15%, 7% and 14%, 7% and 13%, 7% and 12%, 7% and 11%, 7% and 10%, 7% and 9%, 7% and 8%, 8% and 25%, 8% and 24%, 8% and 23%, 8% and 22%, 8% and 21%, 8% and 20%, 8% and 19%, 8% and 18%, 8% and 17%, 8% and 16%, 8% and 15%, 8% and 14%, 8% and 13%, 8% and 12%, 8% and 11%, 8% and 10%, 8% and 9%, 9% and 25%, 9% and 24%, 9% and 23%, 9% and 22%, 9% and 21%, 9% and 20%, 9% and 19%, 9% and 18%, 9% and 17%, 9% and 16%, 9% and 15%, 9% and 14%, 9% and 13%, 9% and 12%, 9% and 11%, 9% and 10%, 10% and 25%, 10% and 24%, 10% and 23%, 10% and 22%, 10% and 21%, 10% and 20%, 10% and 19%, 10% and 18%, 0% and 17%, 10% and 16%, 10% and 15%, 10% and 14%, 10% and 13%, 10% and 12%, 10% and 11%, 11% and 25%, 11% and 24%, 11% and 23%, 11% and 22%, 11% and 21%, 11% and 20%, 11% and 19%, 11% and 18%, 11% and 17%, 11% and 16%, 11% and 15%, 11% and 14%, 11% and 13%, 11% and 12%, 12% and 25%, 12% and 24%, 12% and 23%, 12% and 22%, 12% and 21%, 12% and 20%, 12% and 19%, 12% and 18%, 12% and 17%, 12% and 16%, 12% and 15%, 12% and 14%, 12% and 13%, 13% and 25%, 13% and 24%, 13% and 23%, 13% and 22%, 13% and 21%, 13% and 20%, 13% and 19%, 13% and 18%, 13% and 17%, 13% and 16%, 13% and 15%, 13% and 14%, 14% and 25%, 14% and 24%, 14% and 23%, 14% and 22%, 14% and 21%, 14% and 20%, 14% and 19%, 14% and 18%, 14% and 17%, 14% and 16%, 14% and 15%, 15% and 25%, 15% and 24%, 15% and 23%, 15% and 22%, 15% and 21%, 15% and 20%, 15% and 19%, 15% and 18%, 15% and 17%, 15% and 16%, 16% and 25%, 16% and 24%, 16% and 23%, 16% and 22%, 16% and 21%, 16% and 20%, 16% and 19%, 16% and 18%, 16% and 17%, 17% and 25%, 17% and 24%, 17% and 23%, 17% and 22%, 17% and 21%, 17% and 20%, 17% and 19%, 17% and 18%, 18% and 25%, 18% and 24%, 18% and 23%, 18% and 22%, 18% and 21%, 18% and 20%, 18% and 19%, 19% and 25%, 19% and 24%, 19% and 23%, 19% and 22%, 19% and 21%, 19% and 20%, 20% and 25%, 20% and 24%, 20% and 23%, 20% and 22%, 20% and 21%, 21% and 25%, 21% and 24%, 21% and 23%, 21% and 22%, 22% and 25%, 22% and 24%, 22% and 23%, 23% and 25%, 23% and 24%, or 24% and 25%. In another embodiment, a seed of the Cannabis plants as described herein is provided. In an embodiment, the seed further comprises a trait introduced by backcrossing or genetic transformation. In an embodiment, a Cannabis plant, or part thereof, is provided that includes at least one plant cell, produced by growing the seed. In another embodiment, an F1 hybrid seed produced by crossing the Cannabis plants as described with a different Cannabis plant is provided. In an embodiment, a plant or plant part is provided that is grown from the F1 hybrid seed that comprises at least one cell of the F1 hybrid plant. In another embodiment, a tissue culture of cells produced from the Cannabis plants as described herein is provided. In an embodiment, a plant generated from the tissue culture is provided. In an embodiment, a protoplast produced from the Cannabis plants as described herein is provided. In another embodiment, a Cannabis product produced from the Cannabis plants as described herein is provided. In an embodiment, a method of generating a Cannabis product is provided. In an embodiment, the product is a kief, hashish, bubble hash, an edible product, flower, seed, solvent reduced oil, sludge, e-juice, or tincture.

In an embodiment, a Cannabis plant is provided that comprises a tetrahydrocannabivarin (“THCV”) and cannabidivarin (“CBDV”) combined amount of between 5 and 25%. In an embodiment, the THCV and CBDV combined amount is one or more of between 5% and 24%, 5% and 23%, 5% and 22%, 5% and 21%, 5% and 20%, 5% and 19%, 5% and 18%, 5% and 17%, 5% and 16%, 5% and 15%, 5% and 14%, 5% and 13%, 5% and 12%, 5% and 11%, 5% and 10%, 5% and 9%, 5% and 8%, 5% and 7%, 5% and 6%, 6% and 25%, 6% and 24%, 6% and 23%, 6% and 22%, 6% and 21%, 6% and 20%, 6% and 19%, 6% and 18%, 6% and 17%, 6% and 16%, 6% and 15%, 6% and 14%, 6% and 13%, 6% and 12%, 6% and 11%, 6% and 10%, 6% and 9%, 6% and 8%, 6% and 7%, 7% and 25%, 7% and 24%, 7% and 23%, 7% and 22%, 7% and 21%, 7% and 20%, 7% and 19%, 7% and 18%, 7% and 17%, 7% and 16%, 7% and 15%, 7% and 14%, 7% and 13%, 7% and 12%, 7% and 11%, 7% and 10%, 7% and 9%, 7% and 8%, 8% and 25%, 8% and 24%, 8% and 23%, 8% and 22%, 8% and 21%, 8% and 20%, 8% and 19%, 8% and 18%, 8% and 17%, 8% and 16%, 8% and 15%, 8% and 14%, 8% and 13%, 8% and 12%, 8% and 11%, 8% and 10%, 8% and 9%, 9% and 25%, 9% and 24%, 9% and 23%, 9% and 22%, 9% and 21%, 9% and 20%, 9% and 19%, 9% and 18%, 9% and 17%, 9% and 16%, 9% and 15%, 9% and 14%, 9% and 13%, 9% and 12%, 9% and 11%, 9% and 10%, 10% and 25%, 10% and 24%, 10% and 23%, 10% and 22%, 10% and 21%, 10% and 20%, 10% and 19%, 10% and 18%, 0% and 17%, 10% and 16%, 10% and 15%, 10% and 14%, 10% and 13%, 10% and 12%, 10% and 11%, 11% and 25%, 11% and 24%, 11% and 23%, 11% and 22%, 11% and 21%, 11% and 20%, 11% and 19%, 11% and 18%, 11% and 17%, 11% and 16%, 11% and 15%, 11% and 14%, 11% and 13%, 11% and 12%, 12% and 25%, 12% and 24%, 12% and 23%, 12% and 22%, 12% and 21%, 12% and 20%, 12% and 19%, 12% and 18%, 12% and 17%, 12% and 16%, 12% and 15%, 12% and 14%, 12% and 13%, 13% and 25%, 13% and 24%, 13% and 23%, 13% and 22%, 13% and 21%, 13% and 20%, 13% and 19%, 13% and 18%, 13% and 17%, 13% and 16%, 13% and 15%, 13% and 14%, 14% and 25%, 14% and 24%, 14% and 23%, 14% and 22%, 14% and 21%, 14% and 20%, 14% and 19%, 14% and 18%, 14% and 17%, 14% and 16%, 14% and 15%, 15% and 25%, 15% and 24%, 15% and 23%, 15% and 22%, 15% and 21%, 15% and 20%, 15% and 19%, 15% and 18%, 15% and 17%, 15% and 16%, 16% and 25%, 16% and 24%, 16% and 23%, 16% and 22%, 16% and 21%, 16% and 20%, 16% and 19%, 16% and 18%, 16% and 17%, 17% and 25%, 17% and 24%, 17% and 23%, 17% and 22%, 17% and 21%, 17% and 20%, 17% and 19%, 17% and 18%, 18% and 25%, 18% and 24%, 18% and 23%, 18% and 22%, 18% and 21%, 18% and 20%, 18% and 19%, 19% and 25%, 19% and 24%, 19% and 23%, 19% and 22%, 19% and 21%, 19% and 20%, 20% and 25%, 20% and 24%, 20% and 23%, 20% and 22%, 20% and 21%, 21% and 25%, 21% and 24%, 21% and 23%, 21% and 22%, 22% and 25%, 22% and 24%, 22% and 23%, 23% and 25%, 23% and 24%, or 24% and 25%. In another embodiment, a seed of the Cannabis plants as described herein is provided. In an embodiment, the seed further comprises a trait introduced by backcrossing or genetic transformation. In an embodiment, a Cannabis plant, or part thereof, is provided that includes at least one plant cell, produced by growing the seed. In another embodiment, an F1 hybrid seed produced by crossing the Cannabis plants as described with a different Cannabis plant is provided. In an embodiment, a plant or plant part is provided that is grown from the F1 hybrid seed that comprises at least one cell of the F1 hybrid plant. In another embodiment, a tissue culture of cells produced from the Cannabis plants as described herein is provided. In an embodiment, a plant generated from the tissue culture is provided. In an embodiment, a protoplast produced from the Cannabis plants as described herein is provided. In another embodiment, a Cannabis product produced from the Cannabis plants as described herein is provided. In an embodiment, a method of generating a Cannabis product is provided. In an embodiment, the product is a kief, hashish, bubble hash, an edible product, flower, seed, solvent reduced oil, sludge, e-juice, or tincture.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is an image of a flowering plant having high THCV.

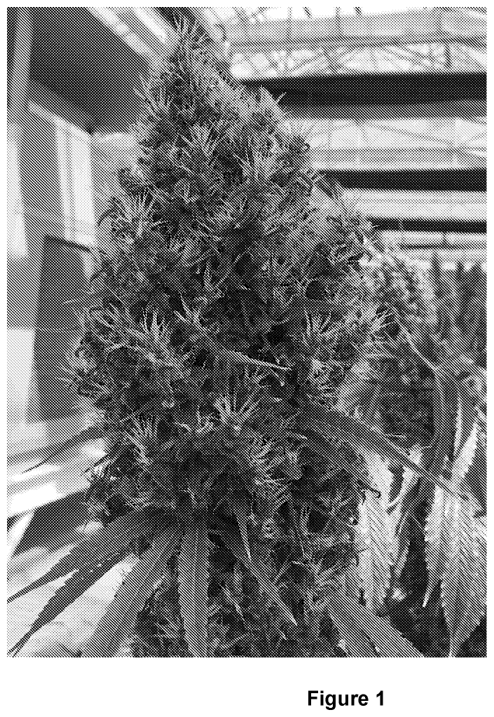

### DETAILED DESCRIPTION OF THE INVENTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The present teachings relate generally to novel Cannabis plants, plant parts, methods, and products having high levels of THCV and/or CBDV.

The terminology used in the disclosure herein is for the purpose of describing particular embodiments only and is not intended to be limiting of the disclosure. As used in the description of the embodiments of the disclosure and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, amount, dose, time, temperature, for example, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs.

### Definitions

The term “acidic cannabinoid” refers to a cannabinoid having one or more carboxylic acid functional groups. Examples of acidic cannabinoids include, but are not limited to, tetrahydrocannabinolic acid (THCA), cannabidiolic acid (CBDA), tetrahydrocannabivarinic acid (THCVA), cannabidivarinic acid (CBDVA), or cannabichromenic acid (CBC). Acidic cannabinoids are frequently the predominant cannabinoids found in raw (i.e., unprocessed) Cannabis plant material.

The term “backcrossing” or “to backcross” refers to the crossing of an F1 hybrid with one of the original parents. A backcross is used to maintain the identity of one parent (species) and to incorporate a particular trait from a second parent (species). The best strategy is to cross the F1 hybrid back to the parent possessing the most desirable traits. Two or more generations of backcrossing may be necessary, but this is practical only if the desired characteristic or trait is present in the F1.

The term “CBDV” means cannabidivarin.

The term “CBDVA” means cannabidivarinic acid.

The term “CBGV” means cannabigerivarin.

The term “CBGVA” means cannabigerivarinic acid.

The term “Cannabis” refers to plants of the genus Cannabis, including Cannabis sativa, Cannabis indica, and Cannabis ruderalis.

The term “Cannabis oil” refers to a mixture of compounds obtained from the extraction of Cannabis plants. Such compounds include, but are not limited to, cannabinoids, terpenes, terpenoids, and other compounds found in the Cannabis plant. The exact composition of Cannabis oil will depend on the strain of Cannabis that is used for extraction, the efficiency and process of the extraction itself, and any additives that might be incorporated to alter the palatability or improve administration of the Cannabis oil.

The term “cannabinoid type I” refers to Total THC: Total CBD ratios, or plants having said ratios, of greater than 3.

The term “cannabinoid type II” refers to Total THC: Total CBD ratios, or plants having said ratios, of between 0.33 and 3.

The term “cannabinoid type III” refers to Total THC: Total CBD ratios, or plants having said ratios, of less than 0.33.

The term “cannabinoid type IV” refers to Total THC less than or equal to 0.5%, Total CBD less than or equal to 0.5%, and CBG levels greater than or equal to 3%, or plants having said percentages.

The term “cell” includes a plant cell, whether isolated, in tissue culture, or incorporated in a plant or plant part.

The term “cross”, “crossing”, “cross pollination” or “cross-breeding” refer to the process by which the pollen of one flower on one plant is applied (artificially or naturally) to the ovule (stigma) of a flower on another plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “cultivar” means a group of similar plants that by structural features and performance (e.g., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain, plant and race are often used interchangeably by plant breeders, agronomists and farmers.

The term “donor plants” refers to the parents of a variety which contains the gene or trait of interest which is desired to be introduced into a second variety (e.g., “recipient plants”).

The term “extract” refers to a solution that has been purged or dehydrated to remove residual solvent. In the methods of the invention, the extract is formed by purging or dehydrating the distillate using any known means in the art.

The term “genotype” refers to the genetic makeup of an individual cell, cell culture, tissue, organism (e.g., a plant), or group of organisms.

A plant is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “hybrid” refers to a variety or cultivar that is the result of a cross of plants of two different varieties. “F1 hybrid” refers to the first generation hybrid, “F2 hybrid” the second generation hybrid, “F3 hybrid” the third generation, and so on. A hybrid refers to any progeny that is either produced or developed.

As used herein, the term “inbreeding” refers to the production of offspring via the mating between relatives. The plants resulting from the inbreeding process are referred to herein as “inbred plants” or “inbreds.”

As used herein, a “landrace” refers to a local variety of a domesticated plant species which has developed largely by natural processes, by adaptation to the natural and cultural environment in which it lives. The development of a landrace may also involve some selection by humans but it differs from a formal breed which has been selectively bred deliberately to conform to a particular formal, purebred standard of traits.

The term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The term “neutral cannabinoid” refers to a cannabinoid without carboxylic acid functional groups. Examples of neutral cannabinoids include, but are not limited to, THC, THCV, CBD, CBG, CBC, and CBN.

The term “offspring” refers to any plant resulting as progeny from a vegetative or sexual reproduction from one or more parent plants or descendants thereof. For instance an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The present disclosure provides ovules and pollens of plants. As used herein when discussing plants, the term “ovule” refers to the female gametophyte, whereas the term “pollen” means the male gametophyte.

The term “plant” refers to whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein. In an embodiment described herein are plants in the genus of Cannabis and plants derived thereof, which can be produced asexual or sexual reproduction.

The term “plant part” or “plant tissue” refers to any part of a plant including but not limited to, an embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen. Plant part may also include certain extracts such as kief, oil, or hash which includes Cannabis trichomes or glands.

The term “progeny” refers to any plant resulting from a vegetative or sexual reproduction from one or more parent plants or descendants thereof. For instance a progeny plant may be obtained by cloning or selfing of a parent plant or by crossing two parent plants and include selfings as well as the F1 or F2 or still further generations. An F1 is a first-generation progeny produced from parents at least one of which is used for the first time as donor of a trait, while offspring of second generation (F2) or subsequent generations (F3, F4, etc.) are specimens produced from selfings of F1's F2's etc. An F1 may thus be (and usually is) a hybrid resulting from a cross between two true breeding parents (true-breeding is homozygous for a trait), while an F2 may be (and usually is) an progeny resulting from self-pollination of said F1 hybrids.

The term “protoplast” as used herein refers to an entire plant cell, excluding the cell wall.

The term “sample” includes a sample from a plant, a plant part, a plant cell, or from a transmission vector, or a soil, water or air sample.

The term “secondary metabolites” as used herein refers to organic compounds that are not directly involved in the normal growth, development, or reproduction of an organism. In other words, loss of secondary metabolites does not result in immediate death of said organism.

The term “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

The term “THCV” means tetrahydrocannabivarin.

The term “THCVA” mean tetrahydrocannabivarinic acid.

The term “tissue culture” refers to a composition comprising isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, suckers and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference

The term “transformant” refers to a cell, tissue or organism that has undergone transformation. The original transformant is designated as “TO” or “TO.” Selfing the TO produces a first transformed generation designated as “T1” or “T1.”

The term “transformation” refers to the transfer of nucleic acid (i.e., a nucleotide polymer) into a cell. As used herein, the term “genetic transformation” refers to the transfer and incorporation of DNA, especially recombinant DNA, into a cell.

The term “varin” as used herein refers to varinolic cannabinoids having three carbon atoms in their alkyl side chain, which may include, but are not limited to, CBCV, CBDV, CBGV, and THCV.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

### Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis strains have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a Cannabis plant contain a single leaflet, with subsequent leaves developing in opposite formation, with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day, and are capable of reaching heights of up to 20 feet. Indoor growth pruning techniques tend to limit Cannabis size through careful pruning of apical or side shoots.

For most cannabinoid producing purposes, only female plants are desired. The presence of male flowers is considered undesirable as pollination is known to reduce the cannabinoid yield, and potentially ruin a crop. For this reason, most Cannabis is grown “sinsemilla” through vegetative (i.e., asexual) propagation. In this way, only female plants are produced and no space is wasted on male plants.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12:R102).

All known strains of Cannabis are wind-pollinated and the fruit is an achene. Most strains of Cannabis are short day plants, with the possible exception of C. sativa subsp. sativa var. spontanea (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the Hemp family (Cannabaceae sensu stricto). Recent phylogenetic studies based on cpDNA restriction site analysis and gene sequencing strongly suggest that the Cannabaceae sensu stricto arose from within the former Celtidaceae family, and that the two families should be merged to form a single monophyletic family, the Cannabaceae sensu lato.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the Cannabis plant (Rudolf Brenneisen, 2007, Chemistry and Analysis of Phytocannabinoids (cannabinoids produced produced by Cannabis) and other Cannabis Constituents, In Marijuana and the Cannabinoids, ElSohly, ed.; incorporated herein by reference) and at least 85 different cannabinoids have been isolated from the plant (El-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42; incorporated herein by reference). The two cannabinoids usually produced in greatest abundance are cannabidiol (CBD) and/or Δ⁹-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

Detection of neutral and acidic forms of cannabinoids are dependent on the detection method utilized. Two popular detection methods are high-performance liquid chromatography (HPLC) and gas chromatography (GC). HPLC separates, identifies, and quantifies different components in a mixture, and passes a pressurized liquid solvent containing the sample mixture through a column filled with a solid adsorbent material. Each molecular component in a sample mixture interacts differentially with the adsorbent material, thus causing different flow rates for the different components and therefore leading to separation of the components. In contrast, GC separates components of a sample through vaporization. The vaporization required for such separation occurs at high temperature. Thus, the main difference between GC and HPLC is that GC involves thermal stress and mainly resolves analytes by boiling points while HPLC does not involve heat and mainly resolves analytes by polarity. The consequence of utilizing different methods for cannabinoid detection therefore is that HPLC is more likely to detect acidic cannabinoid precursors, whereas GC is more likely to detect decarboxylated neutral cannabinoids.

The cannabinoids in Cannabis plants include, but are not limited to, Δ9-Tetrahydrocannabinol (Δ⁹-THC), Δ⁸-Tetrahydrocannabinol (Δ⁸-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), 49-Tetrahydrocannabivarin (THCV), cannabichromevarin (CBCV), and cannabigerovarin (CBGV). See Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes. Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

### Varins

Varins are a type of cannabinoid compounds having three carbon atoms in their alkyl side chain instead of the five carbon atom alkyl side chains more commonly associated with cannabinoids. Two such varins are tetrahydrocannabivarin (THCV) and cannabidivarin (CBDV), which are homologues of tetrahydrocannabinol (THC) and cannabidiol (CBD), respectively.

THCV is a homologue of tetrahydrocannabinol (THC) with a unique pharmacological profile and distinct molecular targets. THCV is a cannabinoid receptor type 1 antagonist and cannabinoid receptor type 2 partial agonist. Δ8-THCV has also been shown to be a CB1 antagonist, an agonist of GPR55 and I-α-lysophosphatidylinositol (LPI), and activator of 5HT1A receptors. THCV promises potential benefits across a broad set of applications.

One such application of the varins described herein is for medical markets. Cannabis strains or extracts with high THCV levels can be used as an agent for anticonvulsant activity, obesity-associated glucose intolerance, appetite suppression, anxiety management for PTSD, diabetic neuropathy, and major neuropathic and pain related pathologies. Another THCV application is an appetite suppressing recreational compound. CBDV has been shown to have anti-epileptic and anticonvulsant activity.

Tetrahydrocannabivarinic acid (THCVA) is the carboxylated precursor to THCV, and the compound present in Cannabis varieties. Cannabidivarinic Acid (CBDVA) is the carboxylated precursor to CBDV, and the compound present in Cannabis varieties. As mentioned herein, phytocannabinoids such as THCV or CBDV are synthesized in the plant as acid forms (e.g., THCVA and CBDVA, respectively), and while some decarboxylation does occur in the plant, it increases significantly post-harvest and the kinetics increase at high temperatures.

In an exemplary embodiment, Cannabis plants are developed using the breeding methods described herein and THCV levels are measured using any of the cannabinoids testing methods described herein. In an embodiment, a Cannabis plant is provided comprising a THCV content between 5% and 25%.

In an embodiment, the THCV content is between 5% and 24%. In an embodiment, the THCV content is between 5% and 23%. In an embodiment, the THCV content is between 5% and 22%. In an embodiment, the THCV content is between 5% and 21%. In an embodiment, the THCV content is between 5% and 20%. In an embodiment, the THCV content is between 5% and 19%. In an embodiment, the THCV content is between 5% and 18%. In an embodiment, the THCV content is between 5% and 17%. In an embodiment, the THCV content is between 5% and 16%. In an embodiment, the THCV content is between 5% and 15%. In an embodiment, the THCV content is between 5% and 14%. In an embodiment, the THCV content is between 5% and 13%. In an embodiment, the THCV content is between 5% and 12%. In an embodiment, the THCV content is between 5% and 11%. In an embodiment, the THCV content is between 5% and 10%. In an embodiment, the THCV content is between 5% and 9%. In an embodiment, the THCV content is between 5% and 8%. In an embodiment, the THCV content is between 5% and 7%. In an embodiment, the THCV content is between 5% and 6%.

In an embodiment, a Cannabis plant is provided comprising a THCV content between 6% and 25%. In an embodiment, the THCV content is between 6% and 24%. In an embodiment, the THCV content is between 6% and 23%. In an embodiment, the THCV content is between 6% and 22%. In an embodiment, the THCV content is between 6% and 21%. In an embodiment, the THCV content is between 6% and 20%. In an embodiment, the THCV content is between 6% and 19%. In an embodiment, the THCV content is between 6% and 18%. In an embodiment, the THCV content is between 6% and 17%. In an embodiment, the THCV content is between 6% and 16%. In an embodiment, the THCV content is between 6% and 15%. In an embodiment, the THCV content is between 6% and 14%. In an embodiment, the THCV content is between 6% and 13%. In an embodiment, the THCV content is between 6% and 12%. In an embodiment, the THCV content is between 6% and 11%. In an embodiment, the THCV content is between 6% and 10%. In an embodiment, the THCV content is between 6% and 9%. In an embodiment, the THCV content is between 6% and 8%. In an embodiment, the THCV content is between 6% and 7%.

In an embodiment, a Cannabis plant is provided comprising a THCV content between 7% and 25%. In an embodiment, the THCV content is between 7% and 24%. In an embodiment, the THCV content is between 7% and 23%. In an embodiment, the THCV content is between 7% and 22%. In an embodiment, the THCV content is between 7% and 21%. In an embodiment, the THCV content is between 7% and 20%. In an embodiment, the THCV content is between 7% and 19%. In an embodiment, the THCV content is between 7% and 18%. In an embodiment, the THCV content is between 7% and 17%. In an embodiment, the THCV content is between 7% and 16%. In an embodiment, the THCV content is between 7% and 15%. In an embodiment, the THCV content is between 7% and 14%. In an embodiment, the THCV content is between 7% and 13%. In an embodiment, the THCV content is between 7% and 12%. In an embodiment, the THCV content is between 7% and 11%. In an embodiment, the THCV content is between 7% and 10%. In an embodiment, the THCV content is between 7% and 9%. In an embodiment, the THCV content is between 7% and 8%.

In an embodiment, a Cannabis plant is provided comprising a THCV content between 8% and 25%. In an embodiment, the THCV content is between 8% and 24%. In an embodiment, the THCV content is between 8% and 23%. In an embodiment, the THCV content is between 8% and 22%. In an embodiment, the THCV content is between 8% and 21%. In an embodiment, the THCV content is between 8% and 20%. In an embodiment, the THCV content is between 8% and 19%. In an embodiment, the THCV content is between 8% and 18%. In an embodiment, the THCV content is between 8% and 17%. In an embodiment, the THCV content is between 8% and 16%. In an embodiment, the THCV content is between 8% and 15%. In an embodiment, the THCV content is between 8% and 14%. In an embodiment, the THCV content is between 8% and 13%. In an embodiment, the THCV content is between 8% and 12%. In an embodiment, the THCV content is between 8% and 11%. In an embodiment, the THCV content is between 8% and 10%. In an embodiment, the THCV content is between 8% and 9%. In an embodiment, a Cannabis plant is provided comprising a THCV content between 9% and 25%. In an embodiment, the THCV content is between 9% and 24%. In an embodiment, the THCV content is between 9% and 23%. In an embodiment, the THCV content is between 9% and 22%. In an embodiment, the THCV content is between 9% and 21%. In an embodiment, the THCV content is between 9% and 20%. In an embodiment, the THCV content is between 9% and 19%. In an embodiment, the THCV content is between 9% and 18%. In an embodiment, the THCV content is between 9% and 17%. In an embodiment, the THCV content is between 9% and 16%. In an embodiment, the THCV content is between 9% and 15%. In an embodiment, the THCV content is between 9% and 14%. In an embodiment, the THCV content is between 9% and 13%. In an embodiment, the THCV content is between 9% and 12%. In an embodiment, the THCV content is between 9% and 11%. In an embodiment, the THCV content is between 9% and 10%.

In an embodiment, a Cannabis plant is provided comprising a THCV content between 10% and 25%. In an embodiment, the THCV content is between 10% and 24%. In an embodiment, the THCV content is between 10% and 23%. In an embodiment, the THCV content is between 10% and 22%. In an embodiment, the THCV content is between 10% and 21%. In an embodiment, the THCV content is between 10% and 20%. In an embodiment, the THCV content is between 10% and 19%. In an embodiment, the THCV content is between 10% and 18%. In an embodiment, the THCV content is between 10% and 17%. In an embodiment, the THCV content is between 10% and 16%. In an embodiment, the THCV content is between 10% and 15%. In an embodiment, the THCV content is between 10% and 14%. In an embodiment, the THCV content is between 10% and 13%. In an embodiment, the THCV content is between 10% and 12%. In an embodiment, the THCV content is between 10% and 11%.

In an embodiment, a Cannabis plant is provided comprising a THCV content between 11% and 25%. In an embodiment, the THCV content is between 11% and 24%. In an embodiment, the THCV content is between 11% and 23%. In an embodiment, the THCV content is between 11% and 22%. In an embodiment, the THCV content is between 11% and 21%. In an embodiment, the THCV content is between 11% and 20%. In an embodiment, the THCV content is between 11% and 19%. In an embodiment, the THCV content is between 11% and 18%. In an embodiment, the THCV content is between 11% and 17%. In an embodiment, the THCV content is between 11% and 16%. In an embodiment, the THCV content is between 11% and 15%. In an embodiment, the THCV content is between 11% and 14%. In an embodiment, the THCV content is between 11% and 13%. In an embodiment, the THCV content is between 11% and 12%.

In an embodiment, a Cannabis plant is provided comprising a THCV content between 12% and 25%. In an embodiment, the THCV content is between 12% and 24%. In an embodiment, the THCV content is between 12% and 23%. In an embodiment, the THCV content is between 12% and 22%. In an embodiment, the THCV content is between 12% and 21%. In an embodiment, the THCV content is between 12% and 20%. In an embodiment, the THCV content is between 12% and 19%. In an embodiment, the THCV content is between 12% and 18%. In an embodiment, the THCV content is between 12% and 17%. In an embodiment, the THCV content is between 12% and 16%. In an embodiment, the THCV content is between 12% and 15%. In an embodiment, the THCV content is between 12% and 14%. In an embodiment, the THCV content is between 12% and 13%.

In an embodiment, a Cannabis plant is provided comprising a THCV content between 13% and 25%. In an embodiment, the THCV content is between 13% and 24%. In an embodiment, the THCV content is between 13% and 23%. In an embodiment, the THCV content is between 13% and 22%. In an embodiment, the THCV content is between 13% and 21%. In an embodiment, the THCV content is between 13% and 20%. In an embodiment, the THCV content is between 13% and 19%. In an embodiment, the THCV content is between 13% and 18%. In an embodiment, the THCV content is between 13% and 17%. In an embodiment, the THCV content is between 13% and 16%. In an embodiment, the THCV content is between 13% and 15%. In an embodiment, the THCV content is between 13% and 14%.

In an embodiment, a Cannabis plant is provided comprising a THCV content between 14% and 25%. In an embodiment, the THCV content is between 14% and 24%. In an embodiment, the THCV content is between 14% and 23%. In an embodiment, the THCV content is between 14% and 22%. In an embodiment, the THCV content is between 14% and 21%. In an embodiment, the THCV content is between 14% and 20%. In an embodiment, the THCV content is between 14% and 19%. In an embodiment, the THCV content is between 14% and 18%. In an embodiment, the THCV content is between 14% and 17%. In an embodiment, the THCV content is between 14% and 16%. In an embodiment, the THCV content is between 14% and 15%.

In an embodiment, a Cannabis plant is provided comprising a THCV content between 15% and 25%. In an embodiment, the THCV content is between 15% and 24%. In an embodiment, the THCV content is between 15% and 23%. In an embodiment, the THCV content is between 15% and 22%. In an embodiment, the THCV content is between 15% and 21%. In an embodiment, the THCV content is between 15% and 20%. In an embodiment, the THCV content is between 15% and 19%. In an embodiment, the THCV content is between 15% and 18%. In an embodiment, the THCV content is between 15% and 17%. In an embodiment, the THCV content is between 15% and 16%.

In an embodiment, a Cannabis plant is provided comprising a THCV content between 16% and 25%. In an embodiment, the THCV content is between 16% and 24%. In an embodiment, the THCV content is between 16% and 23%. In an embodiment, the THCV content is between 16% and 22%. In an embodiment, the THCV content is between 16% and 21%. In an embodiment, the THCV content is between 16% and 20%. In an embodiment, the THCV content is between 16% and 19%. In an embodiment, the THCV content is between 16% and 18%. In an embodiment, the THCV content is between 16% and 17%.

In an embodiment, a Cannabis plant is provided comprising a THCV content between 17% and 25%. In an embodiment, the THCV content is between 17% and 24%. In an embodiment, the THCV content is between 17% and 23%. In an embodiment, the THCV content is between 17% and 22%. In an embodiment, the THCV content is between 17% and 21%. In an embodiment, the THCV content is between 17% and 20%. In an embodiment, the THCV content is between 17% and 19%. In an embodiment, the THCV content is between 17% and 18%.

In an embodiment, a Cannabis plant is provided comprising a THCV content between 18% and 25%. In an embodiment, the THCV content is between 18% and 24%. In an embodiment, the THCV content is between 18% and 23%. In an embodiment, the THCV content is between 18% and 22%. In an embodiment, the THCV content is between 18% and 21%. In an embodiment, the THCV content is between 18% and 20%. In an embodiment, the THCV content is between 18% and 19%.

In an embodiment, a Cannabis plant is provided comprising a THCV content between 19% and 25%. In an embodiment, the THCV content is between 19% and 24%. In an embodiment, the THCV content is between 19% and 23%. In an embodiment, the THCV content is between 19% and 22%. In an embodiment, the THCV content is between 19% and 21%. In an embodiment, the THCV content is between 19% and 20%.

In an embodiment, a Cannabis plant is provided comprising a THCV content between 20% and 25%. In an embodiment, the THCV content is between 20% and 24%. In an embodiment, the THCV content is between 20% and 23%. In an embodiment, the THCV content is between 20% and 22%. In an embodiment, the THCV content is between 20% and 21%.

In an embodiment, a Cannabis plant is provided comprising a THCV content between 21% and 25%. In an embodiment, the THCV content is between 21% and 24%. In an embodiment, the THCV content is between 21% and 23%. In an embodiment, the THCV content is between 21% and 22%.

In an embodiment, a Cannabis plant is provided comprising a THCV content between 22% and 25%. In an embodiment, the THCV content is between 22% and 24%. In an embodiment, the THCV content is between 22% and 23%.

In an embodiment, a Cannabis plant is provided comprising a THCV content between 23% and 25%. In an embodiment, the THCV content is between 23% and 24%.

In an embodiment, a Cannabis plant is provided comprising a THCV content between 24% and 25%. In an embodiment, the Cannabis plant has at least 25% THCV.

In an exemplary embodiment, Cannabis plants are developed using the breeding methods described herein and CBDV levels are measured using any of the cannabinoids testing methods described herein. In an embodiment, a Cannabis plant is provided comprising a CBDV content between 5% and 25%.

In an embodiment, the CBDV content is between 5% and 24%. In an embodiment, the CBDV content is between 5% and 23%. In an embodiment, the CBDV content is between 5% and 22%. In an embodiment, the CBDV content is between 5% and 21%. In an embodiment, the CBDV content is between 5% and 20%. In an embodiment, the CBDV content is between 5% and 19%. In an embodiment, the CBDV content is between 5% and 18%. In an embodiment, the CBDV content is between 5% and 17%. In an embodiment, the CBDV content is between 5% and 16%. In an embodiment, the CBDV content is between 5% and 15%. In an embodiment, the CBDV content is between 5% and 14%. In an embodiment, the CBDV content is between 5% and 13%. In an embodiment, the CBDV content is between 5% and 12%. In an embodiment, the CBDV content is between 5% and 11%. In an embodiment, the CBDV content is between 5% and 10%. In an embodiment, the CBDV content is between 5% and 9%. In an embodiment, the CBDV content is between 5% and 8%. In an embodiment, the CBDV content is between 5% and 7%. In an embodiment, the CBDV content is between 5% and 6%.

In an embodiment, a Cannabis plant is provided comprising a CBDV content between 6% and 25%. In an embodiment, the CBDV content is between 6% and 24%. In an embodiment, the CBDV content is between 6% and 23%. In an embodiment, the CBDV content is between 6% and 22%. In an embodiment, the CBDV content is between 6% and 21%. In an embodiment, the CBDV content is between 6% and 20%. In an embodiment, the CBDV content is between 6% and 19%. In an embodiment, the CBDV content is between 6% and 18%. In an embodiment, the CBDV content is between 6% and 17%. In an embodiment, the CBDV content is between 6% and 16%. In an embodiment, the CBDV content is between 6% and 15%. In an embodiment, the CBDV content is between 6% and 14%. In an embodiment, the CBDV content is between 6% and 13%. In an embodiment, the CBDV content is between 6% and 12%. In an embodiment, the CBDV content is between 6% and 11%. In an embodiment, the CBDV content is between 6% and 10%. In an embodiment, the CBDV content is between 6% and 9%. In an embodiment, the CBDV content is between 6% and 8%. In an embodiment, the CBDV content is between 6% and 7%.

In an embodiment, a Cannabis plant is provided comprising a CBDV content between 7% and 25%. In an embodiment, the CBDV content is between 7% and 24%. In an embodiment, the CBDV content is between 7% and 23%. In an embodiment, the CBDV content is between 7% and 22%. In an embodiment, the CBDV content is between 7% and 21%. In an embodiment, the CBDV content is between 7% and 20%. In an embodiment, the CBDV content is between 7% and 19%. In an embodiment, the CBDV content is between 7% and 18%. In an embodiment, the CBDV content is between 7% and 17%. In an embodiment, the CBDV content is between 7% and 16%. In an embodiment, the CBDV content is between 7% and 15%. In an embodiment, the CBDV content is between 7% and 14%. In an embodiment, the CBDV content is between 7% and 13%. In an embodiment, the CBDV content is between 7% and 12%. In an embodiment, the CBDV content is between 7% and 11%. In an embodiment, the CBDV content is between 7% and 10%. In an embodiment, the CBDV content is between 7% and 9%. In an embodiment, the CBDV content is between 7% and 8%.

In an embodiment, a Cannabis plant is provided comprising a CBDV content between 8% and 25%. In an embodiment, the CBDV content is between 8% and 24%. In an embodiment, the CBDV content is between 8% and 23%. In an embodiment, the CBDV content is between 8% and 22%. In an embodiment, the CBDV content is between 8% and 21%. In an embodiment, the CBDV content is between 8% and 20%. In an embodiment, the CBDV content is between 8% and 19%. In an embodiment, the CBDV content is between 8% and 18%. In an embodiment, the CBDV content is between 8% and 17%. In an embodiment, the CBDV content is between 8% and 16%. In an embodiment, the CBDV content is between 8% and 15%. In an embodiment, the CBDV content is between 8% and 14%. In an embodiment, the CBDV content is between 8% and 13%. In an embodiment, the CBDV content is between 8% and 12%. In an embodiment, the CBDV content is between 8% and 11%. In an embodiment, the CBDV content is between 8% and 10%. In an embodiment, the CBDV content is between 8% and 9%.

In an embodiment, a Cannabis plant is provided comprising a CBDV content between 9% and 25%. In an embodiment, the CBDV content is between 9% and 24%. In an embodiment, the CBDV content is between 9% and 23%. In an embodiment, the CBDV content is between 9% and 22%. In an embodiment, the CBDV content is between 9% and 21%. In an embodiment, the CBDV content is between 9% and 20%. In an embodiment, the CBDV content is between 9% and 19%. In an embodiment, the CBDV content is between 9% and 18%. In an embodiment, the CBDV content is between 9% and 17%. In an embodiment, the CBDV content is between 9% and 16%. In an embodiment, the CBDV content is between 9% and 15%. In an embodiment, the CBDV content is between 9% and 14%. In an embodiment, the CBDV content is between 9% and 13%. In an embodiment, the CBDV content is between 9% and 12%. In an embodiment, the CBDV content is between 9% and 11%. In an embodiment, the CBDV content is between 9% and 10%.

In an embodiment, a Cannabis plant is provided comprising a CBDV content between 10% and 25%. In an embodiment, the CBDV content is between 10% and 24%. In an embodiment, the CBDV content is between 10% and 23%. In an embodiment, the CBDV content is between 10% and 22%. In an embodiment, the CBDV content is between 10% and 21%. In an embodiment, the CBDV content is between 10% and 20%. In an embodiment, the CBDV content is between 10% and 19%. In an embodiment, the CBDV content is between 10% and 18%. In an embodiment, the CBDV content is between 10% and 17%. In an embodiment, the CBDV content is between 10% and 16%. In an embodiment, the CBDV content is between 10% and 15%. In an embodiment, the CBDV content is between 10% and 14%. In an embodiment, the CBDV content is between 10% and 13%. In an embodiment, the CBDV content is between 10% and 12%. In an embodiment, the CBDV content is between 10% and 11%.

In an embodiment, a Cannabis plant is provided comprising a CBDV content between 11% and 25%. In an embodiment, the CBDV content is between 11% and 24%. In an embodiment, the CBDV content is between 11% and 23%. In an embodiment, the CBDV content is between 11% and 22%. In an embodiment, the CBDV content is between 11% and 21%. In an embodiment, the CBDV content is between 11% and 20%. In an embodiment, the CBDV content is between 11% and 19%. In an embodiment, the CBDV content is between 11% and 18%. In an embodiment, the CBDV content is between 11% and 17%. In an embodiment, the CBDV content is between 11% and 16%. In an embodiment, the CBDV content is between 11% and 15%. In an embodiment, the CBDV content is between 11% and 14%. In an embodiment, the CBDV content is between 11% and 13%. In an embodiment, the CBDV content is between 11% and 12%.

In an embodiment, a Cannabis plant is provided comprising a CBDV content between 12% and 25%. In an embodiment, the CBDV content is between 12% and 24%. In an embodiment, the CBDV content is between 12% and 23%. In an embodiment, the CBDV content is between 12% and 22%. In an embodiment, the CBDV content is between 12% and 21%. In an embodiment, the CBDV content is between 12% and 20%. In an embodiment, the CBDV content is between 12% and 19%. In an embodiment, the CBDV content is between 12% and 18%. In an embodiment, the CBDV content is between 12% and 17%. In an embodiment, the CBDV content is between 12% and 16%. In an embodiment, the CBDV content is between 12% and 15%. In an embodiment, the CBDV content is between 12% and 14%. In an embodiment, the CBDV content is between 12% and 13%.

In an embodiment, a Cannabis plant is provided comprising a CBDV content between 13% and 25%. In an embodiment, the CBDV content is between 13% and 24%. In an embodiment, the CBDV content is between 13% and 23%. In an embodiment, the CBDV content is between 13% and 22%. In an embodiment, the CBDV content is between 13% and 21%. In an embodiment, the CBDV content is between 13% and 20%. In an embodiment, the CBDV content is between 13% and 19%. In an embodiment, the CBDV content is between 13% and 18%. In an embodiment, the CBDV content is between 13% and 17%. In an embodiment, the CBDV content is between 13% and 16%. In an embodiment, the CBDV content is between 13% and 15%. In an embodiment, the CBDV content is between 13% and 14%.

In an embodiment, a Cannabis plant is provided comprising a CBDV content between 14% and 25%. In an embodiment, the CBDV content is between 14% and 24%. In an embodiment, the CBDV content is between 14% and 23%. In an embodiment, the CBDV content is between 14% and 22%. In an embodiment, the CBDV content is between 14% and 21%. In an embodiment, the CBDV content is between 14% and 20%. In an embodiment, the CBDV content is between 14% and 19%. In an embodiment, the CBDV content is between 14% and 18%. In an embodiment, the CBDV content is between 14% and 17%. In an embodiment, the CBDV content is between 14% and 16%. In an embodiment, the CBDV content is between 14% and 15%.

In an embodiment, a Cannabis plant is provided comprising a CBDV content between 15% and 25%. In an embodiment, the CBDV content is between 15% and 24%. In an embodiment, the CBDV content is between 15% and 23%. In an embodiment, the CBDV content is between 15% and 22%. In an embodiment, the CBDV content is between 15% and 21%. In an embodiment, the CBDV content is between 15% and 20%. In an embodiment, the CBDV content is between 15% and 19%. In an embodiment, the CBDV content is between 15% and 18%. In an embodiment, the CBDV content is between 15% and 17%. In an embodiment, the CBDV content is between 15% and 16%.

In an embodiment, a Cannabis plant is provided comprising a CBDV content between 16% and 25%. In an embodiment, the CBDV content is between 16% and 24%. In an embodiment, the CBDV content is between 16% and 23%. In an embodiment, the CBDV content is between 16% and 22%. In an embodiment, the CBDV content is between 16% and 21%. In an embodiment, the CBDV content is between 16% and 20%. In an embodiment, the CBDV content is between 16% and 19%. In an embodiment, the CBDV content is between 16% and 18%. In an embodiment, the CBDV content is between 16% and 17%.

In an embodiment, a Cannabis plant is provided comprising a CBDV content between 17% and 25%. In an embodiment, the CBDV content is between 17% and 24%. In an embodiment, the CBDV content is between 17% and 23%. In an embodiment, the CBDV content is between 17% and 22%. In an embodiment, the CBDV content is between 17% and 21%. In an embodiment, the CBDV content is between 17% and 20%. In an embodiment, the CBDV content is between 17% and 19%. In an embodiment, the CBDV content is between 17% and 18%.

In an embodiment, a Cannabis plant is provided comprising a CBDV content between 18% and 25%. In an embodiment, the CBDV content is between 18% and 24%. In an embodiment, the CBDV content is between 18% and 23%. In an embodiment, the CBDV content is between 18% and 22%. In an embodiment, the CBDV content is between 18% and 21%. In an embodiment, the CBDV content is between 18% and 20%. In an embodiment, the CBDV content is between 18% and 19%.

In an embodiment, a Cannabis plant is provided comprising a CBDV content between 19% and 25%. In an embodiment, the CBDV content is between 19% and 24%. In an embodiment, the CBDV content is between 19% and 23%. In an embodiment, the CBDV content is between 19% and 22%. In an embodiment, the CBDV content is between 19% and 21%. In an embodiment, the CBDV content is between 19% and 20%.

In an embodiment, a Cannabis plant is provided comprising a CBDV content between 20% and 25%. In an embodiment, the CBDV content is between 20% and 24%. In an embodiment, the CBDV content is between 20% and 23%. In an embodiment, the CBDV content is between 20% and 22%. In an embodiment, the CBDV content is between 20% and 21%.

In an embodiment, a Cannabis plant is provided comprising a CBDV content between 21% and 25%. In an embodiment, the CBDV content is between 21% and 24%. In an embodiment, the CBDV content is between 21% and 23%. In an embodiment, the CBDV content is between 21% and 22%.

In an embodiment, a Cannabis plant is provided comprising a CBDV content between 22% and 25%. In an embodiment, the CBDV content is between 22% and 24%. In an embodiment, the CBDV content is between 22% and 23%.

In an embodiment, a Cannabis plant is provided comprising a CBDV content between 23% and 25%. In an embodiment, the CBDV content is between 23% and 24%.

In an embodiment, a Cannabis plant is provided comprising a CBDV content between 24% and 25%. In an embodiment, the Cannabis plant has at least 25% CBDV.

In an exemplary embodiment, Cannabis plants are developed using the breeding methods described herein and THCV and CBDV combined levels are measured using any of the cannabinoids testing methods described herein. In an embodiment, a Cannabis plant is provided comprising a THCV and CBDV combined content of between 5% and 25%.

In an embodiment, the THCV and CBDV combined content is between 5% and 24%. In an embodiment, the THCV and CBDV combined content is between 5% and 23%. In an embodiment, the THCV and CBDV combined content is between 5% and 22%. In an embodiment, the THCV and CBDV combined content is between 5% and 21%. In an embodiment, the THCV and CBDV combined content is between 5% and 20%. In an embodiment, the THCV and CBDV combined content is between 5% and 19%. In an embodiment, the THCV and CBDV combined content is between 5% and 18%. In an embodiment, the THCV and CBDV combined content is between 5% and 17%. In an embodiment, the THCV and CBDV combined content is between 5% and 16%. In an embodiment, the THCV and CBDV combined content is between 5% and 15%. In an embodiment, the THCV and CBDV combined content is between 5% and 14%. In an embodiment, the THCV and CBDV combined content is between 5% and 13%. In an embodiment, the THCV and CBDV combined content is between 5% and 12%. In an embodiment, the THCV and CBDV combined content is between 5% and 11%. In an embodiment, the THCV and CBDV combined content is between 5% and 10%. In an embodiment, the THCV and CBDV combined content is between 5% and 9%. In an embodiment, the THCV and CBDV combined content is between 5% and 8%. In an embodiment, the THCV and CBDV combined content is between 5% and 7%. In an embodiment, the THCV and CBDV combined content is between 5% and 6%.

In an embodiment, a Cannabis plant is provided comprising a THCV and CBDV combined content of between 6% and 25%. In an embodiment, the THCV and CBDV combined content is between 6% and 24%. In an embodiment, the THCV and CBDV combined content is between 6% and 23%. In an embodiment, the THCV and CBDV combined content is between 6% and 22%. In an embodiment, the THCV and CBDV combined content is between 6% and 21%. In an embodiment, the THCV and CBDV combined content is between 6% and 20%. In an embodiment, the THCV and CBDV combined content is between 6% and 19%. In an embodiment, the THCV and CBDV combined content is between 6% and 18%. In an embodiment, the THCV and CBDV combined content is between 6% and 17%. In an embodiment, the THCV and CBDV combined content is between 6% and 16%. In an embodiment, the THCV and CBDV combined content is between 6% and 15%. In an embodiment, the THCV and CBDV combined content is between 6% and 14%. In an embodiment, the THCV and CBDV combined content is between 6% and 13%. In an embodiment, the THCV and CBDV combined content is between 6% and 12%. In an embodiment, the THCV and CBDV combined content is between 6% and 11%. In an embodiment, the THCV and CBDV combined content is between 6% and 10%. In an embodiment, the THCV and CBDV combined content is between 6% and 9%. In an embodiment, the THCV and CBDV combined content is between 6% and 8%. In an embodiment, the THCV and CBDV combined content is between 6% and 7%.

In an embodiment, a Cannabis plant is provided comprising a THCV and CBDV combined content of between 7% and 25%. In an embodiment, the THCV and CBDV combined content is between 7% and 24%. In an embodiment, the THCV and CBDV combined content is between 7% and 23%. In an embodiment, the THCV and CBDV combined content is between 7% and 22%. In an embodiment, the THCV and CBDV combined content is between 7% and 21%. In an embodiment, the THCV and CBDV combined content is between 7% and 20%. In an embodiment, the THCV and CBDV combined content is between 7% and 19%. In an embodiment, the THCV and CBDV combined content is between 7% and 18%. In an embodiment, the THCV and CBDV combined content is between 7% and 17%. In an embodiment, the THCV and CBDV combined content is between 7% and 16%. In an embodiment, the THCV and CBDV combined content is between 7% and 15%. In an embodiment, the THCV and CBDV combined content is between 7% and 14%. In an embodiment, the THCV and CBDV combined content is between 7% and 13%. In an embodiment, the THCV and CBDV combined content is between 7% and 12%. In an embodiment, the THCV and CBDV combined content is between 7% and 11%. In an embodiment, the THCV and CBDV combined content is between 7% and 10%. In an embodiment, the THCV and CBDV combined content is between 7% and 9%. In an embodiment, the THCV and CBDV combined content is between 7% and 8%.

In an embodiment, a Cannabis plant is provided comprising a THCV and CBDV combined content of between 8% and 25%. In an embodiment, the THCV and CBDV combined content is between 8% and 24%. In an embodiment, the THCV and CBDV combined content is between 8% and 23%. In an embodiment, the THCV and CBDV combined content is between 8% and 22%. In an embodiment, the THCV and CBDV combined content is between 8% and 21%. In an embodiment, the THCV and CBDV combined content is between 8% and 20%. In an embodiment, the THCV and CBDV combined content is between 8% and 19%. In an embodiment, the THCV and CBDV combined content is between 8% and 18%. In an embodiment, the THCV and CBDV combined content is between 8% and 17%. In an embodiment, the THCV and CBDV combined content is between 8% and 16%. In an embodiment, the THCV and CBDV combined content is between 8% and 15%. In an embodiment, the THCV and CBDV combined content is between 8% and 14%. In an embodiment, the THCV and CBDV combined content is between 8% and 13%. In an embodiment, the THCV and CBDV combined content is between 8% and 12%. In an embodiment, the THCV and CBDV combined content is between 8% and 11%. In an embodiment, the THCV and CBDV combined content is between 8% and 10%. In an embodiment, the THCV and CBDV combined content is between 8% and 9%.

In an embodiment, a Cannabis plant is provided comprising a THCV and CBDV combined content of between 9% and 25%. In an embodiment, the THCV and CBDV combined content is between 9% and 24%. In an embodiment, the THCV and CBDV combined content is between 9% and 23%. In an embodiment, the THCV and CBDV combined content is between 9% and 22%. In an embodiment, the THCV and CBDV combined content is between 9% and 21%. In an embodiment, the THCV and CBDV combined content is between 9% and 20%. In an embodiment, the THCV and CBDV combined content is between 9% and 19%. In an embodiment, the THCV and CBDV combined content is between 9% and 18%. In an embodiment, the THCV and CBDV combined content is between 9% and 17%. In an embodiment, the THCV and CBDV combined content is between 9% and 16%. In an embodiment, the THCV and CBDV combined content is between 9% and 15%. In an embodiment, the THCV and CBDV combined content is between 9% and 14%. In an embodiment, the THCV and CBDV combined content is between 9% and 13%. In an embodiment, the THCV and CBDV combined content is between 9% and 12%. In an embodiment, the THCV and CBDV combined content is between 9% and 11%. In an embodiment, the THCV and CBDV combined content is between 9% and 10%.

In an embodiment, a Cannabis plant is provided comprising a THCV and CBDV combined content of between 10% and 25%. In an embodiment, the THCV and CBDV combined content is between 10% and 24%. In an embodiment, the THCV and CBDV combined content is between 10% and 23%. In an embodiment, the THCV and CBDV combined content is between 10% and 22%. In an embodiment, the THCV and CBDV combined content is between 10% and 21%. In an embodiment, the THCV and CBDV combined content is between 10% and 20%. In an embodiment, the THCV and CBDV combined content is between 10% and 19%. In an embodiment, the THCV and CBDV combined content is between 10% and 18%. In an embodiment, the THCV and CBDV combined content is between 10% and 17%. In an embodiment, the THCV and CBDV combined content is between 10% and 16%. In an embodiment, the THCV and CBDV combined content is between 10% and 15%. In an embodiment, the THCV and CBDV combined content is between 10% and 14%. In an embodiment, the THCV and CBDV combined content is between 10% and 13%. In an embodiment, the THCV and CBDV combined content is between 10% and 12%. In an embodiment, the THCV and CBDV combined content is between 10% and 11%.

In an embodiment, a Cannabis plant is provided comprising a THCV and CBDV combined content of between 11% and 25%. In an embodiment, the THCV and CBDV combined content is between 11% and 24%. In an embodiment, the THCV and CBDV combined content is between 11% and 23%. In an embodiment, the THCV and CBDV combined content is between 11% and 22%. In an embodiment, the THCV and CBDV combined content is between 11% and 21%. In an embodiment, the THCV and CBDV combined content is between 11% and 20%. In an embodiment, the THCV and CBDV combined content is between 11% and 19%. In an embodiment, the THCV and CBDV combined content is between 11% and 18%. In an embodiment, the THCV and CBDV combined content is between 11% and 17%. In an embodiment, the THCV and CBDV combined content is between 11% and 16%. In an embodiment, the THCV and CBDV combined content is between 11% and 15%. In an embodiment, the THCV and CBDV combined content is between 11% and 14%. In an embodiment, the THCV and CBDV combined content is between 11% and 13%. In an embodiment, the THCV and CBDV combined content is between 11% and 12%.

In an embodiment, a Cannabis plant is provided comprising a THCV and CBDV combined content of between 12% and 25%. In an embodiment, the THCV and CBDV combined content is between 12% and 24%. In an embodiment, the THCV and CBDV combined content is between 12% and 23%. In an embodiment, the THCV and CBDV combined content is between 12% and 22%. In an embodiment, the THCV and CBDV combined content is between 12% and 21%. In an embodiment, the THCV and CBDV combined content is between 12% and 20%. In an embodiment, the THCV and CBDV combined content is between 12% and 19%. In an embodiment, the THCV and CBDV combined content is between 12% and 18%. In an embodiment, the THCV and CBDV combined content is between 12% and 17%. In an embodiment, the THCV and CBDV combined content is between 12% and 16%. In an embodiment, the THCV and CBDV combined content is between 12% and 15%. In an embodiment, the THCV and CBDV combined content is between 12% and 14%. In an embodiment, the THCV and CBDV combined content is between 12% and 13%.

In an embodiment, a Cannabis plant is provided comprising a THCV and CBDV combined content of between 13% and 25%. In an embodiment, the THCV and CBDV combined content is between 13% and 24%. In an embodiment, the THCV and CBDV combined content is between 13% and 23%. In an embodiment, the THCV and CBDV combined content is between 13% and 22%. In an embodiment, the THCV and CBDV combined content is between 13% and 21%. In an embodiment, the THCV and CBDV combined content is between 13% and 20%. In an embodiment, the THCV and CBDV combined content is between 13% and 19%. In an embodiment, the THCV and CBDV combined content is between 13% and 18%. In an embodiment, the THCV and CBDV combined content is between 13% and 17%. In an embodiment, the THCV and CBDV combined content is between 13% and 16%. In an embodiment, the THCV and CBDV combined content is between 13% and 15%. In an embodiment, the THCV and CBDV combined content is between 13% and 14%.

In an embodiment, a Cannabis plant is provided comprising a THCV and CBDV combined content of between 14% and 25%. In an embodiment, the THCV and CBDV combined content is between 14% and 24%. In an embodiment, the THCV and CBDV combined content is between 14% and 23%. In an embodiment, the THCV and CBDV combined content is between 14% and 22%. In an embodiment, the THCV and CBDV combined content is between 14% and 21%. In an embodiment, the THCV and CBDV combined content is between 14% and 20%. In an embodiment, the THCV and CBDV combined content is between 14% and 19%. In an embodiment, the THCV and CBDV combined content is between 14% and 18%. In an embodiment, the THCV and CBDV combined content is between 14% and 17%. In an embodiment, the THCV and CBDV combined content is between 14% and 16%. In an embodiment, the THCV and CBDV combined content is between 14% and 15%.

In an embodiment, a Cannabis plant is provided comprising a THCV and CBDV combined content of between 15% and 25%. In an embodiment, the THCV and CBDV combined content is between 15% and 24%. In an embodiment, the THCV and CBDV combined content is between 15% and 23%. In an embodiment, the THCV and CBDV combined content is between 15% and 22%. In an embodiment, the THCV and CBDV combined content is between 15% and 21%. In an embodiment, the THCV and CBDV combined content is between 15% and 20%. In an embodiment, the THCV and CBDV combined content is between 15% and 19%. In an embodiment, the THCV and CBDV combined content is between 15% and 18%. In an embodiment, the THCV and CBDV combined content is between 15% and 17%. In an embodiment, the THCV and CBDV combined content is between 15% and 16%.

In an embodiment, a Cannabis plant is provided comprising a THCV and CBDV combined content of between 16% and 25%. In an embodiment, the THCV and CBDV combined content is between 16% and 24%. In an embodiment, the THCV and CBDV combined content is between 16% and 23%. In an embodiment, the THCV and CBDV combined content is between 16% and 22%. In an embodiment, the THCV and CBDV combined content is between 16% and 21%. In an embodiment, the THCV and CBDV combined content is between 16% and 20%. In an embodiment, the THCV and CBDV combined content is between 16% and 19%. In an embodiment, the THCV and CBDV combined content is between 16% and 18%. In an embodiment, the THCV and CBDV combined content is between 16% and 17%.

In an embodiment, a Cannabis plant is provided comprising a THCV and CBDV combined content of between 17% and 25%. In an embodiment, the THCV and CBDV combined content is between 17% and 24%. In an embodiment, the THCV and CBDV combined content is between 17% and 23%. In an embodiment, the THCV and CBDV combined content is between 17% and 22%. In an embodiment, the THCV and CBDV combined content is between 17% and 21%. In an embodiment, the THCV and CBDV combined content is between 17% and 20%. In an embodiment, the THCV and CBDV combined content is between 17% and 19%. In an embodiment, the THCV and CBDV combined content is between 17% and 18%.

In an embodiment, a Cannabis plant is provided comprising a THCV and CBDV combined content of between 18% and 25%. In an embodiment, the THCV and CBDV combined content is between 18% and 24%. In an embodiment, the THCV and CBDV combined content is between 18% and 23%. In an embodiment, the THCV and CBDV combined content is between 18% and 22%. In an embodiment, the THCV and CBDV combined content is between 18% and 21%. In an embodiment, the THCV and CBDV combined content is between 18% and 20%. In an embodiment, the THCV and CBDV combined content is between 18% and 19%.

In an embodiment, a Cannabis plant is provided comprising a THCV and CBDV combined content of between 19% and 25%. In an embodiment, the THCV and CBDV combined content is between 19% and 24%. In an embodiment, the THCV and CBDV combined content is between 19% and 23%. In an embodiment, the THCV and CBDV combined content is between 19% and 22%. In an embodiment, the THCV and CBDV combined content is between 19% and 21%. In an embodiment, the THCV and CBDV combined content is between 19% and 20%.

In an embodiment, a Cannabis plant is provided comprising a THCV and CBDV combined content of between 20% and 25%. In an embodiment, the THCV and CBDV combined content is between 20% and 24%. In an embodiment, the THCV and CBDV combined content is between 20% and 23%. In an embodiment, the THCV and CBDV combined content is between 20% and 22%. In an embodiment, the THCV and CBDV combined content is between 20% and 21%.

In an embodiment, a Cannabis plant is provided comprising a THCV and CBDV combined content of between 21% and 25%. In an embodiment, the THCV and CBDV combined content is between 21% and 24%. In an embodiment, the THCV and CBDV combined content is between 21% and 23%. In an embodiment, the THCV and CBDV combined content is between 21% and 22%.

In an embodiment, a Cannabis plant is provided comprising a THCV and CBDV combined content of between 22% and 25%. In an embodiment, the THCV and CBDV combined content is between 22% and 24%. In an embodiment, the THCV and CBDV combined content is between 22% and 23%.

In an embodiment, a Cannabis plant is provided comprising a THCV and CBDV combined content of between 23% and 25%. In an embodiment, the THCV and CBDV combined content is between 23% and 24%.

In an embodiment, a Cannabis plant is provided comprising a THCV and CBDV combined content of between 24% and 25%. In an embodiment, the Cannabis plant has at least 25% THCV and CBDV combined.

### Cannabis Breeding

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars. The plants described herein can be used to produce new plant varieties. In some embodiments, the plants are used to develop new, unique, and superior varieties or hybrids with desired phenotypes.

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011), each of which is herein incorporated by reference in its entirety for all purposes.

Pedigree selection, where both single plant selection and mass selection practices are employed, may be used for the generating varieties as described herein. Pedigree selection, also known as the “Vilmorin system of selection,” is described in Fehr, Walter; Principles of Cultivar Development, Volume I, Macmillan Publishing Co., which is hereby incorporated by reference. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has been advanced from the F2 to the desired level of inbreeding, the plants from which lines are derived will each trace to different F₂ individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Molecular markers can also be used in breeding programs. Molecular markers can be designed and made, based on the genome of the plants of the present application. Non-limiting examples of molecular markers can be Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Single Nucleotide Polymorphisms (SNPs), Amplified Fragment Length Polymorphisms (AFLPs), and Simple Sequence Repeats (SSRs), which are also referred to as Microsatellites, etc. Methods of developing molecular markers and their applications are described by Avise (Molecular markers, natural history, and evolution, Publisher: Sinauer Associates, 2004, ISBN 0878930418, 9780878930418), Srivastava et al. (Plant biotechnology and molecular markers, Publisher: Springer, 2004, ISBN1402019114, 9781402019111), and Vienne (Molecular markers in plant genetics and biotechnology, Publisher: Science Publishers, 2003), each of which is incorporated by reference in its entirety for all purposes. Molecular markers can be used in molecular marker assisted breeding. For example, the molecular markers can be utilized to monitor the transfer of the genetic material. The transferred genetic material is a gene of interest, such as genes that contribute to one or more favorable agronomic phenotypes when expressed in a plant cell, a plant part, or a plant.

Isozyme Electrophoresis and RFLPs have been widely used to determine genetic composition. Shoemaker and Olsen, (Molecular Linkage Map of Soybean (Glycine max) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety for all purposes. Cannabis genome has been sequenced (Bakel et al., The draft genome and transcriptome of Cannabis sativa, Genome Biology, 12 (10): R102, 2011). Molecular markers for Cannabis plants are described in Datwyler et al. (Genetic variation in hemp and marijuana (Cannabis sativa L.) according to amplified fragment length polymorphisms, J Forensic Sci. 2006 March; 51 (2): 371-5), Pinarkara et al., (RAPD analysis of seized marijuana (Cannabis sativa L.) in Turkey, Electronic Journal of Biotechnology, 12 (1), 2009), Hakki et al., (Inter simple sequence repeats separate efficiently hemp from marijuana (Cannabis sativa L.), Electronic Journal of Biotechnology, 10 (4), 2007), Datwyler et al., (Genetic Variation in Hemp and Marijuana (Cannabis sativa L.) According to Amplified Fragment Length Polymorphisms, J Forensic Sci, March 2006, 51 (2): 371-375), Gilmore et al. (Isolation of microsatellite markers in Cannabis sativa L. (marijuana), Molecular Ecology Notes, 3 (1): 105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety for all purposes.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

### Methods of Use

The present invention provides methods of using the Cannabis plants or any parts, any compositions, or any chemicals derived from said plants of the present invention. Cannabis oil extracts can be used in the manufacture of a pharmaceutical composition or for a medicament for treating a number of conditions.

The plants can also be used for non-medical purposes. In some embodiments the specialty Cannabis plants of the present invention can be used for recreational purposes. In some embodiments, the specialty Cannabis plants of the present invention can be used for industrial purposes. In some embodiments, the plants are used for producing food, oil, wax, resin, rope, cloth, pulp, fiber, feed for livestock, construction material, plastic and composite materials, paper, jewelry, water and soil purification materials, weed control materials, cultivation materials, textiles, clothing, biodegradable plastics, body products, health food and biofuel.

### Tissue Culture

Further reproduction of the variety can occur by tissue culture and regeneration. As is well known in the art, tissue culture of Cannabis can be used for the in vitro regeneration of a Cannabis plant. Tissue culture of various tissues of Cannabis and regeneration of plants therefrom is well known and widely published. For example, reference may be had to Teng et al., HortScience. 1992, 27:9, 1030-1032 Teng et al., HortScience. 1993, 28:6, 669-1671, Zhang et al., Journal of Genetics and Breeding. 1992, 46:3, 287-290, Webb et al., Plant Cell Tissue and Organ Culture. 1994, 38:1, 77-79, Curtis et al., Journal of Experimental Botany. 1994, 45:279, 1441-1449, Nagata et al., Journal for the American Society for Horticultural Science. 2000, 125:6, 669-672. It is clear from the literature that the state of the art is such that these methods of obtaining plants are, and were, “conventional” in the sense that they are routinely used and have a very high rate of success. Thus, another aspect of this invention is to provide cells which upon growth and differentiation produce Cannabis plants having the physiological and morphological characteristics of variety PBI-0227-CMV.

### Products

In an embodiment a Cannabis extract or product is disclosed. The product may be any product known in the Cannabis arts, and can include, but is not limited to, a kief, hashish, bubble hash, an edible product, flower, seed, solvent reduced oil, sludge, e-juice, or tincture. As used herein, Cannabis sludges are solvent-free Cannabis extracts made via multigas extraction including the refrigerant 134A, butane, iso-butane and propane in a ratio that delivers a very complete and balanced extraction of cannabinoids and essential oils.

Compositions for pulmonary administration also include, but are not limited to, dry powder compositions consisting of the powder of a Cannabis oil described herein, which may be developed using lyophilization, and the powder of a suitable carrier and/or lubricant. The compositions for pulmonary administration can be inhaled from any suitable dry powder inhaler device known to a person skilled in the art. In certain instances, the compositions may be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, maltodextrin, lecithin, agarose, xanthan gum, guar gum, sorbitol, cellulose (e.g., ethyl cellulose, microcrystalline cellulose), glycine, pectin, polyacrylates and/or calcium hydrogen phosphate, calcium sulfate, (b) lubricants; e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium or potassium salt, or effervescent mixtures; (e) wetting agents, e.g., sodium lauryl sulfate, and/or (f) absorbents, colorants, flavors and sweeteners. Tablets can be either uncoated or coated according to methods known in the art. The excipients described herein can also be used for preparation of buccal dosage forms and sublingual dosage forms (e.g., films and lozenges) as described, for example, in U.S. Pat. Nos. 5,981,552 and 8,475,832. Formulation in chewing gums as described, for example, in U.S. Pat. No. 8,722,022, is also contemplated.

Further preparations for oral administration can take the form of, for example, solutions, syrups, suspensions, and toothpastes. Liquid preparations for oral administration can be prepared by conventional means with pharmaceutically acceptable additives, for example, suspending agents, for example, sorbitol syrup, cellulose derivatives, or hydrogenated edible fats; emulsifying agents, for example, lecithin, xanthan gum, or acacia; non-aqueous vehicles, for example, almond oil, sesame oil, hemp seed oil, fish oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate.

Typical formulations for topical administration include creams, ointments, sprays, lotions, hydrocolloid dressings, and patches, as well as eye drops, ear drops, and deodorants. Cannabis oils can be administered via transdermal patches as described, for example, in U.S. Pat. Appl. Pub. No. 2015/0126595 and U.S. Pat. No. 8,449,908. Formulation for rectal or vaginal administration is also contemplated. The Cannabis oils can be formulated, for example, us suppositories containing conventional suppository bases such as cocoa butter and other glycerides as described in U.S. Pat. Nos. 5,508,037 and 4,933,363. Compositions can contain other solidifying agents such as shea butter, beeswax, kokum butter, mango butter, ilipe butter, tamanu butter, carnauba wax, emulsifying wax, soy wax, castor wax, rice bran wax, and candelila wax. Compositions can further include clays (e.g., Bentonite, French green clays, Fuller's earth, Rhassoul clay, white kaolin clay) and salts (e.g., sea salt, Himalayan pink salt, and magnesium salts such as Epsom salt).

The compositions set forth herein can be formulated for parenteral administration by injection, for example, by bolus injection or continuous infusion. Formulations for injection can be presented in unit dosage form, for example, in ampoules or in multi-dose containers, optionally with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure, buffers, and/or other ingredients. Alternatively, the compositions can be in powder form for reconstitution with a suitable vehicle, for example, a carrier oil, before use. In addition, the compositions may also contain other therapeutic agents or substances.

The compositions can be prepared according to conventional mixing, granulating, and/or coating methods, and contain from about 0.1 to about 75%, preferably from about 1 to about 50%, of the Cannabis oil extract. In general, subjects receiving a Cannabis oil composition orally are administered doses ranging from about 1 to about 2000 mg of Cannabis oil. A small dose ranging from about 1 to about 20 mg can typically be administered orally when treatment is initiated, and the dose can be increased (e.g., doubled) over a period of days or weeks until the maximum dose is reached.

### Examples

Aspects of the present teachings can be further understood in light of the following examples, which should not be construed as limiting the scope of the present teachings in any way.

The practice of the present teachings employ, unless otherwise indicated, conventional methods of protein chemistry, biochemistry, recombinant DNA techniques and pharmacology, within the skill of the art. Such techniques are explained fully in the literature. See, e.g., T. Creighton, Proteins: Structures and Molecular Properties, 1993, W. Freeman and Co.; A. Lehninger, Biochemistry, Worth Publishers, Inc. (current addition); J. Sambrook et al., Molecular Cloning: A Laboratory Manual, 2nd Edition, 1989; Methods In Enzymology, S. Colowick and N. Kaplan, eds., Academic Press, Inc.; Remington's Pharmaceutical Sciences, 18th Edition, 1990, Mack Publishing Company, Easton, Pa.; Carey and Sundberg, Advanced Organic Chemistry, Vols. A and B, 3rd Edition, 1992, Plenum Press.

The practice of the present teachings also employ, unless otherwise indicated, conventional methods of statistical analysis, within the skill of the art. Such techniques are explained fully in the literature. See, e.g., J. Little and D. Rubin, Statistical Analysis with Missing Data, 2nd Edition 2002, John Wiley and Sons, Inc., NJ; M. Pepe, The Statistical Evaluation of Medical Tests for Classification and Prediction (Oxford Statistical Science Series) 2003, Oxford University Press, Oxford, UK; X. Zhoue et al., Statistical Methods in Diagnostic Medicine 2002, John Wiley and Sons, Inc., NJ; T. Hastie et. al, The Elements of Statistical Learning: Data Mining, Inference, and Prediction, Second Edition 2009, Springer, N.Y.; W. Cooley and P. Lohnes, Multivariate procedures for the behavioral science 1962, John Wiley and Sons, Inc. NY; E. Jackson, A User's Guide to Principal Components 2003, John Wiley and Sons, Inc., NY.

This example describes the development of Cannabis plants having elevated THCV levels. All breeding, cultivation, and chemotype analyses were conducted by Progressive Plant Research, Inc. under contract for Phylos Bioscience, Inc.

Cannabis plants having elevated THCV levels were sourced and obtained, and crosses were conducted to further elevate THCV levels. The plants are crossed to produce new varieties that are selected to further elevate THCV levels. Progeny are chemically analyzed to select those having elevated THCV levels, and then either (1) backcrossed to the high-yielding THCV parent to ensure additional progeny can be selected for increased THCV production, or (2) selfed to fix loci associated with THCV levels to ensure additional progeny can be selected for increased THCV production. Marker-assisted selection may be used further to identify candidate progeny expected to have elevated THCV levels for subsequent crosses to ensure selection of progeny having the highest THCV levels. All chemo-analyses were conducted using High Performance Liquid Chromatography to detect the cannabinoids described herein.

Chemical analyses of the strains described herein will be carried out using standard chemical separation techniques well known to those skilled in the arts using extended cannabinoids test analysis. The results of Table 1 provide THCVA, Total THC, and Total THCV levels of three samples from a developed cultivar having elevated THCV levels.

TABLE 1
Chemotype THCV analyses of different cultivars.

| | | | | Time | | Total | Total |
|---|---|---|---|---|---|---|---|
| Tissue Type | PCT Serial | Cultivar | GID | Point* | THCVA | THC | THCV |
| dried flower | C-DZAKDP-R | 21TP1A-101-40 | 6493478 | 83 | 15.6 | 11.9 | 14.0 |
| dried flower | C-SRDTUM-R | 22TP1A-41-22 | 8705543 | 41 | 18.56 | 5.51 | 16.54 |

*Time Point = days since the switch to 12:12 light cycle that initiates flowering. Flower sample was taken at maturity.

Table 2 describes a developed cultivar having high levels of different varins. Cultivar 21TP1A-101-40 was chemotyped by taking a sample from the main cola at day 83 after initiation of the 12:12 light cycle, which initiates flowering. Trichomes were 65% cloudy and 1% amber, indicative of full maturity at sampling.

This cultivar is of cannabinoid type I with virtually no CBD produced (Table 2).

TABLE 2
Cannabinoid data for 21TP1A-101-40
(day 83 after flowering onset).

| Cannabinoid | % wt |
|---|---|
| THCVA | 15.6 |
| THCV | 0.28 |
| Total THCV | 14.0 |
| CBDVA | 0.1 |
| CBDV | <LOD |
| Total CBDV | 0.09 |
| Total THCV + Total CBDV (=Total Varins) | 14.0 |
| Total THC + Total CBD (=Total Cannabinoids) | 11.9 |
| Total Varins/Total Cannabinoids | 1.18 |
| THCA | 13.3 |
| D9-THC | 0.2 |
| Total THC | 11.9 |
| CBDA | <LOD |
| CBD | <LOD |
| Total CBD | <LOD |
| THC:CBD ratio | Inf |

Table 3 describes a developed cultivar having high levels of different varins. Cultivar 22TP1A-41-22 was chemotyped by taking a sample from the main cola at day 83 after initiation of the 12:12 light cycle, which initiates flowering.

TABLE 3
Cannabinoid data for 21TP1A-41-22
(day 41 after flowering onset).

| Cannabinoid | % wt |
|---|---|
| THCVA | 18.56 |
| THCV | 0.26 |
| Total THCV | 16.54 |
| CBDVA | 0.23 |
| CBDV | <LOD |
| Total CBDV | 0.2 |
| Total THCV + Total CBDV (=Total Varins) | 16.74 |
| THCA | 6.29 |
| D9-THC | <LOD |
| Total THC | 5.51 |
| CBDA | 0.18 |
| CBD | <LOD |
| Total CBD | 0.16 |
| THC:CBD ratio | 34.4375 |

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

1. A Cannabis plant wherein the plant comprises:
a) a tetrahydrocannabivarin (“THCV”) amount of between 5% and 25%;
b) a combined tetrahydrocannabivarin (“THCV”) and cannabidivarin (“CBDV”) amount of between 5% and 25%; or
c) a cannabidivarin (“CBDV”) amount of between 5% and 25%.

2. The Cannabis plant of claim 1 wherein:
a) the THCV amount is between 12% and 25%;
b) the combined THCV and CBDV amount is between 12% and 25%; or
c) the cannabidivarin (“CBDV”) amount is between 12% and 25%.

3. A seed of the Cannabis plant of claim 1.

4. The seed of claim 3, further comprising a trait introduced by backcrossing or genetic transformation.

5. A Cannabis plant, or part thereof, including at least one plant cell, produced by growing the seed of claim 3.

6. An F1 hybrid seed produced by crossing the Cannabis plant of claim 1 with a different Cannabis plant.

7. An F1 hybrid plant grown from the seed of claim 6, or a plant part thereof, the plant part comprising at least one cell of the F1 hybrid plant.

8. A tissue culture of cells produced from the Cannabis plant of claim 1.

9. A Cannabis plant generated from the tissue culture of claim 8.

10. A protoplast produced from the Cannabis plant of claim 1.

11. A method of generating a processed Cannabis product comprising the use of a plant of claim 1.

12. A Cannabis product produced from the Cannabis plant of claim 1.

13. The product of claim 12 wherein the product is a kief, hashish, bubble hash, an edible product, flower, seed, solvent reduced oil, sludge, e-juice, or tincture.

14-39. (canceled)

40. The Cannabis plant of claim 1, wherein:
a) the THCV amount is between 14% and 18%; or
b) the combined THCV and CBDV amount is between 14% and 18%.

41. The Cannabis plant of claim 1, wherein the plant is cultivar 21TP1A-101-40, 22TP1A-41-22, or a progeny thereof.
